(19) United States
(12) Patent Application Publication
(10) Pub. No.: US 2023/0010958 A1
(43) Pub. Date: Jan. 12, 2023

(54) CANNABINOID AND LYCOPENE ANTI-INFLAMMATORY SYNERGISTIC COMBINATIONS

(71) Applicant: LYCORED LTD., Be’er Sheva (IL)

(72) Inventors: Tal OFFER, Givat Ada (IL); Tanya SEDLOV, Beer Sheva (IL); Etgar LEVY-NISSENBAUM, Ramat HaSharon (IL)

(73) Assignee: LYCORED LTD., Be’er Sheva (IL)

(21) Appl. No.: 17/780,149

(22) PCT Filed: Nov. 26, 2020
(86) PCT No.: PCT/IL2020/051223

§ 371 Date: May 26, 2022

Related U.S. Application Data
(60) Provisional application No. 62/940,293, filed on Nov. 26, 2019.

(51) Int. Cl. A61K 31/05; A61K 36/81; A61K 9/00; A61P 29/00

(52) U.S. Cl. CPC A61K 31/05; A61K 9/0053; A61K 36/81; A61P 29/00

(57) ABSTRACT

The present invention is directed to a composition comprising a cannabinoid and lycopene, kits comprising same, and methods of using same, such as for treating a subject afflicted with inflammation.

### CROSS-REFERENCE TO RELATED APPLICATIONS

This application claims the benefit of priority to U.S. Provisional Pat. Application No. 62/940,293, titled “CANNABINOID AND LYCOPENE ANTI-INFLAMMATORY SYNERGISTIC COMBINATIONS”, filed Nov. 26, 2019, the contents of which are incorporated herein by reference in their entirety.

### FIELD OF THE INVENTION

The invention is directed to anti-inflammatory compositions comprising a cannabinoid and lycopene.

### BACKGROUND

The inflammatory process, which forms an important part of the non-specific immune system, is characterized by a complex set of chemical and cellular changes that are essential for host defense in the face of microbial agents and other potentially harmful environmental factors. However, in many cases, inflammation may be triggered inappropriately, and/or may persist to a degree which becomes harmful to the host. In such cases, there may be a need to inhibit or prevent the development of one or more aspects of the inflammatory process, in particular, in cases of non-infectious inflammatory diseases.

A very large number of different chemical mediators have been shown to be involved in the development and control of the inflammatory process. Recent studies by a number of different laboratories have implicated nitric oxide (NO) as an important modulator of a variety of acute and chronic inflammatory disorders, including various types of arthritis, gastro-intestinal diseases, inflammatory conditions of the central nervous system and certain forms of asthma. Consequently, it has been proposed that inhibition of NO production could provide a useful therapeutic mechanism for the treatment and/or management of these inflammatory disorders. Furthermore, inhibition of NO synthesis has also been shown to be useful in some conditions or states that are not primarily inflammatory in nature. Thus, for example, inhibition of NO synthesis has been found to reduce glucose uptake into limb tissue in individuals with Type 2 diabetes during exercise.

The in vivo production of NO is mediated by a family of nitric oxide synthase (NOS) enzymes, including inducible-nitric oxide synthase (I—NOS), which is activated by many different immunological stimuli including lipopolysaccharide (LPS), interferon gamma and interleukin 1 (IL—1).

Several other compounds, including a number of natural products, have also been shown to inhibit NO production. The latter group includes compounds such as lutein and lycopene. However, the efficacy and potency of many of the natural product NO inhibitors have proven to be not particularly high. A need therefore exists for improved NO production-inhibiting compositions of natural origin.

Another highly important inflammatory mediator is the tumor necrosis factor-alpha (TNF-α), which is a cytokine produced by a variety of cell types including macrophages, neutrophils and lymphocytes. TNF-α occupies a key position in the early stage of the inflammatory process and is responsible for stimulating the production of other factors such as nuclear factor κB which in turn causes activation of a wide range of pro-inflammatory genes. Thus, in view of its key pro-inflammatory role, TNF-α is clearly an important potential therapeutic target for anti-inflammatory agents.

### SUMMARY

The following embodiments and aspects thereof are described and illustrated in conjunction with systems, tools and methods which are meant to be exemplary and illustrative, not limiting in scope.

According to a first aspect, there is provided a composition comprising a cannabinoid and lycopene.

According to another aspect, there is provided a pharmaceutical composition, comprising the composition of the invention and a pharmaceutically acceptable carrier.

According to another aspect, there is provided a method for treating a subject afflicted with inflammation, comprising administering to the subject a therapeutically effective amount of the composition of the invention.

According to another aspect, there is provided a kit comprising: (a) lycopene; (b) a cannabinoid; and (c) mixing instructions for the lycopene and the cannabinoid.

In some embodiments, the molar ratio (m:m) of lycopene to cannabinoid is from 50:1 (m:m) to 1:10 (m:m).

In some embodiments, the cannabinoid is selected from the group consisting of: tetrahydrocannabinol (THC), iso-tetrahydrocannabinol- type (iso-THC), tetrahydrocannabinolic acid (THCA), cannabidiol (CBD), cannabidiolic acid (CBDA), cannabinol (CBN), cannabinolic acid (CBNA), cannabinol methyl ether (CBNM), cannabinol-C4 (CBN—C4), cannabinol-CZ (CBN—C2), cannabiorcol (CBN—C1), cannabinodiol (CBND), cannabigerol (CBG), cannabigerolic acid (CBGA), cannabigerolic acid monomethyl ether (CBGAM), cannabigerol monomethyl ether (CBGM), cannabigerovarinic acid (CBGVA), cannabichromene (CBC), cannabichromanon (CBCN), cannabichromenic acid (CBCA), cannabichromevarin (CBCV), cannabichromevarinic acid (CBCVA), tetrahydrocannabivarin (THCV), cannabidivarin (CBDV), cannabielsoin (CBE), cannabielsoic acid A (CBEA—A), cannabielsoic acid B (CBEA—B), cannabigerovarin (CBGV), cannabidiolic acid (CBDA), cannabidiol monomethyl ether (CBDM), cannabidiol-C4 (CBD—C4), cannabidivarinic acid (CBDVA), and cannabidiorcol (CBD—C1), cannabicyclol (CBL), cannabicyclolic acid (CBLA), cannabicyclovarin (CBLV), cannabitriol, cannabitriolvarin (CBTV), ethoxy-cannabitiolvarin (CBTVE), cannabivarin (CBV), cannabidivarin (CBVD), cannabitriolvarin (CBTV), ethoxy-cannabitiolvarin (CBTVE), cannabifuran (CBF), dehydrocannabifuran (DCBF), and cannabiripsol (CBR), or any combination thereof.

In some embodiments, the cannabinoid is selected from the group consisting of: CBD, (-)-7-hydroxy-CBD, (-)-CBD-7-oic acid, dimethylheptyl homologue of (-)-7-hydroxy-CBD, dimethylheptyl homologue of (-)-CBD-7-oic acid, or any combination thereof.

In some embodiments, the cannabinoid is CBD or CBDA.

In some embodiments, the composition comprises lycopene and CBD in a m:m ratio ranging from 30:1 (m:m) to 10:1 (m:m).

In some embodiments, the composition comprises lycopene and CBDA in a m:m ratio ranging from 1:5 (m:m) to 1:1 (m:m).

In some embodiments, the composition further comprises: phytoene, phytofluene, beta carotene, tocopherol, a phytosterol, astaxanthin, lutein, a terpene, a polyphenol, or any combination thereof.

In some embodiments, the composition further comprises: phytoene, phytofluene, or any combination thereof.

In some embodiments, the composition is an oral composition.

In some embodiments, the cannabinoid is present as a highly purified extract of Cannabis.

In some embodiments, the cannabinoid is a synthetically produced cannabinoid.

In some embodiments, the pharmaceutical composition is for use in treatment of inflammation in a subject in need thereof.

In some embodiments, the treating comprises reducing or inhibiting production of nitric oxide in the subject.

In some embodiments, the kit comprises instructions for administering lycopene and a cannabinoid in a m:m ratio ranging from 50:1 (m:m) to 1:10 (m:m).

In some embodiments, the cannabinoid comprises CBD.

In some embodiments, the cannabinoid comprises CBDA.

In some embodiments, the kit is for use in treatment of inflammation in a subject in need thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by study of the following detailed description.

### BRIEF DESCRIPTION OF THE FIGURES

FIG. 1 includes a vertical bar graph showing the effect of cannabidiol (CBD) on nitric oxide (NO) production. Macrophages were stimulated, and NO production in the presence of increasing CBD concentrations is presented.

FIG. 2 includes a vertical bar graph showing the effect of CBD on cell viability. MTT assay was performed in the presence of increasing CBD concentrations. Gray vertical line indicates that incubating cells in the presence of CBD at a concentration of 25 µM or more led to significant cell toxicity.

FIG. 3 includes a vertical bar graph showing the effect of lycopene (0.5 µM, 1 µM, and 2 µM) on NO production.

FIG. 4 includes a vertical bar graph showing the effect of CBD (0.05 µM, 0.1 µM, and 0.5 µM) on NO production.

FIG. 5 includes a vertical bar graph showing a combined anti-inflammatory effect of lycopene and CBD. Cells were supplemented with both lycopene (0.5 µM, 1 µM, and 2 µM) and CBD (0.05 µM). A significant synergistic anti-inflammatory effect was observed for lycopene at a concentration ranging from 0.5 µM to 1 µM and 0.05 µM CBD.

FIG. 6 includes a vertical bar graph showing a combined anti-inflammatory effect of lycopene and CBD. Cells were supplemented with both lycopene (0.5 µM, 1 µM, and 2 µM) and CBD (0.1 µM). A significant synergistic anti-inflammatory effect was observed for lycopene at a concentration ranging from 1 µM to 2 µM and 0.1 µM CBD.

FIG. 7 includes a vertical bar graph showing a combined anti-inflammatory effect of lycopene and CBD. Cells were supplemented with both lycopene (0.5 µM, 1 µM, and 2.5 µM) and CBD (0.1 µM). A significant synergistic anti-inflammatory effect was observed for lycopene at a concentration ranging from 1 µM to 2.5 µM and 0.1 µM CBD.

FIGS. 8A-8B include vertical bar graphs showing a combined anti-inflammatory effect of lycopene and CBDA. (8A) A vertical bar graph showing the effect of CBDA (2.5 µM, 5 µM, and 10 µM) on NO production. (8B) Cells were supplemented with or without lycopene (1 µM) and with or without CBDA (0.625 µM, 1.25 µM, and 2.5 µM). A significant synergistic anti-inflammatory effect was observed for lycopene at a concentration of 1 µM and 2.5 µM CBDA.

### DETAILED DESCRIPTION

In one embodiment, the present invention provides a composition comprising a cannabinoid and a carotenoid. In one embodiment, the present invention provides a composition comprising a cannabinoid and lycopene. In one embodiment, the present invention provides a composition comprising CBD and lycopene. In one embodiment, the present invention provides a composition comprising CBDA and lycopene.

In one embodiment, lycopene is extracted from a plant or plant material. In one embodiment, lycopene is extracted from a tomato plant or a tomato material. In another embodiment, lycopene is chemically synthesized. In another embodiment, lycopene is biologically synthesized and/or is biosynthetic lycopene.

As used herein, the term “biosynthetic lycopene” refers to lycopene being produced by microbes, such as by means of fermentation.

In some embodiments, the composition comprises a tomato product. In some embodiments, the composition comprises a tomato plant extract. In another embodiment, the composition comprises LYC-O-MATO® (LycoRed Ltd., Be’er Sheva, Israel), which is described in U.S. Pat. No. 5,837,311, the specification of which is incorporated herein by reference in its entirety.

As used herein, the term “cannabinoid” refers to a chemical compound that shows direct or indirect activity at a cannabinoid receptor (CNR). There are two main cannabinoid receptors, CNR1 (also known as CB1) and CNR2 (also known as CB2). Other receptors that have been indicated as having cannabinoid activity include the GPR55, GPR18, and TRPV1 receptors.

In some embodiments, the cannabinoid is chemically synthesized. In some embodiments, the cannabinoid is a synthetically produced cannabinoid. In some embodiments, the cannabinoid is extracted from a plant or a plant part (e.g., “phytocannabinoids” that occur in a plant species or are derived from a cannabis plant species). In some embodiments, the cannabinoid is present as a highly purified extract of Cannabis. In some embodiments, the cannabinoid is produced by microbial fermentation or an equivalent biosynthetic methodology.

As used herein, the term “synthetically produced” refers to the cannabinoid being produced by or synthesized by man. In some embodiments, synthetic comprises “man-made”. In some embodiments, synthetic comprises cultured, grown, processed, manipulated, or any combination thereof, in-vitro.

In some embodiments, synthetic comprises chemically synthesized. In some embodiments, synthetic comprises biologically synthesized. In some embodiments, biologically synthesized comprises synthesis utilizing a cell or a cell-free system. In some embodiments, a cell-free system comprises a system comprising at least one cellular compartment and/or specific proteins and/or molecules, e.g., macromolecules, capable of producing and/or secreting a cannabinoid, in vitro. In some embodiments, the system does not comprise a cell. In some embodiments, the system does not comprise a living cell. In some embodiments, the system is devoid of a cell.

As used herein, the term “biosynthetic cannabinoid” encompasses any cannabinoid being produced by cells in an in vitro culture and/or environment, such as bioreactor, fermenter, or any equivalent thereto, which would be apparent to one of ordinary skill in the art as suitable for culturing cells, e.g., such as for producing a cannabinoid. In some embodiments, the cells producing the biosynthetic cannabinoid are plant cells. In some embodiments, the cells producing the biosynthetic cannabinoid are microorganism cells. In some embodiments, the biosynthetic cannabinoid is produced by and/or secreted from a plant cell cultured under suitable conditions. In some embodiments, the biosynthetic cannabinoid is produced by and/or secreted from a microorganism cultured under suitable conditions. Ins some embodiments, the microorganism is selected from: bacteria, a fungus, a yeast, or any combination thereof. In some embodiments, the plant cell comprises a plant cell line, a primary plant cell culture, a primary plant organ culture, or any combination thereof. In some embodiments, the cell is an isolated cell. In some embodiments, cell is an intact, naive, wildtype, normal, or any combination thereof, of a cell. In some embodiments, the cell is a genetically and/or genomically modified cell. In some embodiments, the cell is genetically and/or genomically modified so as to produce and/or secret a cannabinoid.

In some embodiments, the biosynthetic cannabinoid comprises a cannabinoid produced by fermentation.

Methods for genetically and/or genomically modifying a cell so as to produce and/or secret a cannabinoid are common and would be apparent to one of ordinary skill in the art. Non-limiting examples for such methods include, but are noy limited to, introduction of genes and/or equivalent nucleic acid material encoding at least one gene product capable of producing and/or secreting a cannabinoid. Non-limiting examples of enzymes involved in production of endocannabinoids include, but are not limited to, Abh4 (α/β-hydrolase 4); lyso-PLD (lyso-phospholipase D); sPLA2 (secretory phospholipase A2), PLC (phospholipase C); NAPE-PLD (phospholipase D selective for N-acyl-phosphatidylethanolamines); and NAT (trans-N-acyltransferase), among others.

According to some embodiments, the cannabinoid is selected from: tetrahydrocannabinol (THC), iso-tetrahydrocannabinol (iso-THC), tetrahydrocannabinolic acid (THCA), cannabidiol (CBD), cannabidiolic acid (CBDA), cannabinol (CBN), cannabinolic acid (CBNA), cannabinol methyl ether (CBNM), cannabinol-C4 (CBN—C4), cannabinol-CZ (CBN—C2), cannabiorcol (CBN—C1), cannabinodiol (CBND), cannabigerol (CBG), cannabigerolic acid (CBGA), cannabigerolic acid monomethyl ether (CBGAM), cannabigerol monomethyl ether (CBGM), cannabigerovarinic acid (CBGVA), cannabichromene (CBC), cannabichromanon (CBCN), cannabichromenic acid (CBCA), cannabichromevarin (CBCV), cannabichromevarinic acid (CBCVA), tetrahydrocannabivarin (THCV), cannabidivarin (CBDV), cannabielsoin (CBE), cannabielsoic acid A (CBEA—A), cannabielsoic acid B (CBEA—B), cannabigerovarin (CBGV), cannabidiolic acid (CBDA), cannabidiol monomethyl ether (CBDM), cannabidiol-C4 (CBD—C4), cannabidivarinic acid (CBDVA), and cannabidiorcol (CBD—C1), cannabicyclol (CBL), cannabicyclolic acid (CBLA), cannabicyclovarin (CBLV), cannabitriol, cannabitriolvarin (CBTV), ethoxy-cannabitiolvarin (CBTVE), cannabivarin (CBV), cannabidivarin (CBVD), cannabitriol, cannabitriolvarin (CBTV), ethoxy-cannabitiolvarin (CBTVE), cannabifuran (CBF), dehydrocannabifuran (DCBF), cannabiripsol (CBR), or any combination thereof.

As used herein, the term “cannabinoid” encompasses a plurality of cannabinoids. In some embodiments, a plurality of cannabinoids comprises: at least 2 cannabinoids, at least 3 cannabinoids, at least 4 cannabinoids, at least 5 cannabinoids, at least 6 cannabinoids, or at least 10 cannabinoids, or any value and range therebetween. Each possibility represents a separate embodiment of the invention. In some embodiments, the plurality of cannabinoids comprises: 1 to 5 cannabinoids, 2 to 12 cannabinoids, 3 to 8 cannabinoids, 4 to 15 cannabinoids, or 2 to 10 cannabinoids. Each possibility represents a separate embodiment of the invention.

In some embodiments, a plurality of cannabinoids comprises all naturally occurring cannabinoids (phytocannabinoids). In some embodiments, the plurality of cannabinoids comprises all chemically synthetized cannabinoids. In some embodiments, the plurality of cannabinoids comprises one or more naturally occurring cannabinoids, and one or more chemically synthetized cannabinoids.

In some embodiments, the composition of the invention comprises cannabidiol (CBD).

In some embodiments, the cannabinoid comprises or is CBD.

As used herein the terms “cannabidiol” or “CBD” refer to the cannabinoid which constitutes up to 40% of Cannabis sativa extracts, and is recognized as a major nonpsychoactive cannabinoid, with a remarkable lack of any cognitive and psychoactive actions. CBD has potent anti-inflammatory and immunosuppressive effects. CBD has been shown to inhibit cancer cell growth and to reduce anxiety and nausea. CBD, also termed 2-[(6R)-3-Methyl-6-prop-l-en-2-yl- lcyclohex-2-envyl]-5pentylbenzene-l,3-diol, has the molecular formula of C₂₁H₃₀O₂.

The cannabinoid, for CBD can be provided to the composition described herein in a purified form (e.g., above 90% purity, e.g., synthetic forms) or in a cannabis essential oil (e.g., obtained from trichome extract). The oil can be extracted from a single strain of a cannabis or from a plurality of strains (or genetic backgrounds), wherein the cannabis genetic background is selected according to the intended use.

In some embodiments, the cannabinoid comprises or is CBDA.

In some embodiments, the cannabinoid is selected from CBD, CBDA, or a combination thereof. In some embodiments, the cannabinoid is CBD or CBDA.

As used herein, the terms “Cannabidiolic acid” or “CBDA” refer to an acid precursor of CBD.

In some embodiments, the composition comprises a cannabis plant extract. In some embodiments, cannabis is hemp. In some embodiments, a cannabinoid and or terpene as described herein is extracted and/or isolated from hemp.

As used herein, the cannabis extract can be derived from any strain selected from: Ringo’s Gift, or Harle-Tsu (both by Southern Humboldt Seed Collective), or ACDC strain, or Cannatonic strain, or Dieseltonic or Hammershark (all four by Resin Seeds), or Charlotte’s web (by Charlotte’s Web Holdings), or Felina 32, or Santhica 27, or Futura 75, or Antal, or Kompolti Hybrid-TC, or Kompolti, or KC Dóra (all seven by Hempoint, s.r.o.), or Avidekel, or Metatron or Michael, or Rephael (all four by Tikun olam), or EpiOne Oil (by Better), or Tachllta Till (by Seach), or Remedy (by Remedy), or Berry Blossom, or Queen Dream, or Cherry blossom, or Cobbler Hemp, or Wife Hemp, or Chardonnay Hemp or Cherry Wine all seven by Blue Forest Farms), or Elektra, or Lifter (both by Oregon CBD), or Fermion, or Gliana, or Jubileu, or Monoica, or Carmagnola, or Tisza, or Markant, or Fewdora 17, or Silvana, or Dacia, or CS Selected Carmagnola, or KC Zuzana, or Ratza, or Finola, or Eletta Campana, or Tiborszallasi.

In another embodiment, a composition as described herein further comprises phytoene. In another embodiment, a composition as described herein further comprises phytofluene. In another embodiment, a composition as described herein further comprises beta-carotene. In another embodiment, a composition as described herein further comprises tocopherol. In another embodiment, a composition as described herein further comprises a phytosterol. In another embodiment, a composition as described herein further comprises astaxanthin. In some embodiments, the composition further comprises a terpene. In some embodiments, a composition as described herein further comprises a polyphenol.

In another embodiment, any one of: phytoene, phytofluene, beta-carotene, tocopherol, phytosterol, terpene, and polyphenol, are derived from a plant or plant material, such as, but not limited to a tomato. In another embodiment, any one of: phytoene, phytofluene, beta-carotene, tocopherol, phytosterol, terpene, and polyphenol, are produced synthetically.

In another embodiment, a phytosterol can be a combination of phytosterols.

In another embodiment, a terpene can be a combination of terpenes.

In another embodiment, a polyphenol can be a combination of polyphenols.

In some embodiments, the molar concentration per molar concentration ratio (m:m) of lycopene to a cannabinoid is from 50:1 (m:m) to 1:10 (m:m), 50:1 (m:m) to 1:5 (m:m), 50:1 (m:m) to 1:2.5 (m:m), 50:1 (m:m) to 1:1 (m:m), 40:1 (m:m) to 1:5 (m:m), 30:1 (m:m) to 1:2 (m:m), 25:1 (m:m) to 1:2 (m:m), 20:1 (m:m) to 1:2.5 (m:m), 15:1 (m:m) to 1:1 (m:m), 10:1 (m:m) to 1:1.5 (m:m), 10:1 (m:m) to 1:3 (m:m), or 10:1 (m:m) to 1:1 (m:m). Each possibility represents a separate embodiment of the invention.

In some embodiments, the m:m ratio of lycopene to a cannabinoid is from 10:1 (m:m) to 20:1. In some embodiments, the m:m ratio of lycopene to a cannabinoid is 10:1 (m:m). In some embodiments, the m:m ratio of lycopene to a cannabinoid is to 25:1.

In some embodiments, the m:m of lycopene to CBD is from 50:1 (m:m) to 5:1 (m:m), 40:1 (m:m) to 5:1 (m:m), 30:1 (m:m) to 5:1 (m:m), 20:1 (m:m) to 5:1 (m:m), 10:1 (m:m) to 5:1 (m:m), 7.5:1 (m:m) to 4:1 (m:m), 5:1 (m:m) to 1:1 (m:m), 25:1 (m:m) to 10:1 (m:m), or 20:1 (m:m) to 10:1 (m:m). Each possibility represents a separate embodiment of the invention.

In some embodiments, the m:m ratio of lycopene to CBD is 10:1 (m:m). In some embodiments, the m:m ratio of lycopene to CBD is to 20:1 (m:m). In some embodiments, the m:m ratio of lycopene to CBD is to 25:1 (m:m).

In some embodiments, the m:m of lycopene to CBDA is from 1:1 (m:m) to 1:50 (m:m), 1:1 (m:m) to 1:40 (m:m), 1:1 (m:m) to 1:30 (m:m), 1:1 (m:m) to 1:20 (m:m), 1:1 (m:m) to 1:15 (m:m), 1:1 (m:m) to 1:10 (m:m), 1:1 (m:m) to 1:5 (m:m), or 1:1 (m:m) to 1:4 (m:m), 1:1 (m:m) to 1:3 (m:m), 1:1 (m:m) to 1:2.5 (m:m), or 1:1 (m:m) to 1:2 (m:m). Each possibility represents a separate embodiment of the invention.

In some embodiments, the m:m ratio of lycopene to CBDA is 1:1.5 (m:m). In some embodiments, the m:m ratio of lycopene to CBD is to 1:2 (m:m). In some embodiments, the m:m ratio of lycopene to CBD is to 1:2.5 (m:m). In some embodiments, the m:m ratio of lycopene to CBD is to 1:3 (m:m).

In another embodiment, the weight per weight ratio of the cannabinoid to lycopene is from 1:99 (w/w) to 99:1 (w/w). In another embodiment, the weight per weight ratio of the cannabinoid to lycopene is from 1:80 (w/w) to 80:1 (w/w). In another embodiment, the weight per weight ratio of the cannabinoid to lycopene is from 1:55 (w/w) to 55:1 (w/w). In another embodiment, the weight per weight ratio of the cannabinoid to lycopene is from 1:30 (w/w) to 30:1 (w/w). In another embodiment, the weight per weight ratio of the cannabinoid to lycopene is from 1:10 (w/w) to 10:1 (w/w).

In some embodiments, the composition comprises lycopene at a concentration of at least 0.5 µM, at least 1 µM, at least 1.5 µM, at least 2 µM, at least 3 µM, at least 4 µM, at least 5 µM, at least 6 µM, at least 7 µM, at least 8 µM, or at least 9 µM, or any value and range therebetween. Each possibility represents a separate embodiment of the invention.

In some embodiments, the composition comprises lycopene at a concentration of 0.01 µM to 1,000 µM. In some embodiments, the composition comprises lycopene at a concentration of 0.5 µM to 10 µM.

In another embodiment, a composition as described comprises from 2.5 to 15 mg lycopene. In another embodiment, a composition as described comprises 5 mg lycopene. In another embodiment, a composition as described comprises from 5 to 10 mg lycopene.

In some embodiments, the composition comprises a cannabinoid at a concentration of at least 0.05 µM, at least 0.07 µM, at least 0.08 µM, at least 0.09 µM, at least 0.1 µM, at least 0.2 µM, at least 0.3 µM, or at least 0.4 µM, or any value and range therebetween. Each possibility represents a separate embodiment of the invention.

In some embodiments, the composition comprises a cannabinoid at a concentration of 0.01 µM to 1 µM. In some embodiments, the composition comprises a cannabinoid at a concentration of 0.05 µM to 0.5 µM.

In another embodiment, a composition as described comprises 0.1 to 1 mg of a cannabinoid. In another embodiment, a composition as described comprises 0.5 to 20 mg of a cannabinoid. In another embodiment, a composition as described comprises 0.01 to 2 mg of a cannabinoid. In another embodiment, a composition as described comprises 10 to 100 mg of a cannabinoid.

In another embodiment, a composition of the invention further comprises zeaxanthin. In another embodiment, a composition of the invention further comprises lutein. In another embodiment, lutein comprises (3R,3′R,6′R)-beta,epsilon-carotene-3,3′-diol. In another embodiment, lutein is a plant lutein. In another embodiment, lutein is a chemically synthesized lutein. In another embodiment, lutein is a tomato lutein. In another embodiment, lutein is marigold lutein. In another embodiment, lutein is provided as a marigold extract.

In some embodiments, a composition as described herein inhibits the production and/or secretion of inflammatory mediators and cytokines which play important roles in the pathogenesis of a vast number of mammalian inflammatory diseases. In another embodiment, a composition of the invention is causing an immediate, efficient, or synergistic inhibition of LPS-induced internal superoxide production leading to a marked decrease in ERK and nuclear factor kappa B (NF-kB) activation or any combination thereof.

In another embodiment, the present invention is directed to a method for treating a subject afflicted with inflammation.

In another embodiment, the method of the invention comprises the step of administering to a subject afflicted with inflammation a therapeutically effective amount of a composition comprising a cannabinoid and lycopene.

In another embodiment, the method comprises inhibiting the production, release, or both, of proinflammatory cytokines, such as but not limited to TNF-alpha by macrophages and monocytes at inflammatory sites.

In one embodiment, the inflammation is an inherent part of a disease state.

In another embodiment, the disease is selected from: rheumatoid arthritis, Crohn’s disease, ulcerative colitis, irritable bowel syndrome (IBS), septic shock syndrome, atherosclerosis, juvenile rheumatoid arthritis, psoriatic arthritis, osteoarthritis, refractory rheumatoid arthritis, chronic non-rheumatoid arthritis, osteoporosis/bone resorption, endotoxic shock, ischemia-reperfusion injury, coronary heart disease, vasculitis, amyloidosis, multiple sclerosis, sepsis, chronic recurrent uveitis, hepatitis C virus infection, malaria, ulcerative colitis, cachexia, plasmocytoma, endometriosis, Behcet’s disease, Wegener’s granulomatosis, an autoimmune disease, ankylosing spondylitis, common variable immunodeficiency (CVID), chronic graft-versus-host disease, trauma and transplant rejection, adult respiratory distress syndrome, pulmonary fibrosis, recurrent ovarian cancer, a lymphoproliferative disease, refractory multiple myeloma, myeloproliferative disorder, diabetes, juvenile diabetes, meningitis, skin delayed type hypersensitivity disorders, Alzheimer’s disease, systemic lupus erythematosus, or any other clinical condition which is inherently associated or depends on an inflammatory process. Each possibility represents a separate embodiment of the invention.

In another embodiment, the present invention provides that treating a subject afflicted with inflammation is inhibiting the production of an anti-inflammatory cytokine, a glucocorticoid, an anti-inflammatory neuropeptide, a lipid inflammation mediator, or a combination thereof. In another embodiment, the present invention provides that treating a subject afflicted with inflammation is inhibiting the production of nitric oxide (NO), prostaglandin E (PGE), tumor necrosis factor alpha (TNF-alpha), or any combination thereof at a site of inflammation. In another embodiment, the present invention provides that treating a subject afflicted with inflammation is inhibiting the production of NO, PGE, TNF-alpha, or any combination thereof by macrophages. In another embodiment, the present invention provides that treating a subject afflicted with inflammation is inhibiting the recruitment of neutrophils to the site of inflammation. In another embodiment, the present invention provides that treating a subject afflicted with inflammation is inhibiting neutrophils activation at the site of inflammation. In another embodiment, PGE is PGE2 (prostaglandin E2).

In some embodiments, the subject is a mammal. In some embodiments, the subject is a human subject.

In some embodiments, a composition as described herein has an anti-inflammatory effect. In some embodiments, a composition as described herein has a synergistic anti-inflammatory effect. In some embodiments, a composition as described herein is an oral composition. In some embodiments, a composition as described herein further comprises a pharmaceutically or a nutraceutically acceptable excipient.

In some embodiments, there is provided a pharmaceutical composition comprising the composition of the invention and a pharmaceutically acceptable carrier.

In one embodiment, the composition is a pharmaceutical composition. In one embodiment, the composition is a nutraceutical composition. In another embodiment, the composition is incorporated into a foodstuff or a beverage.

In some embodiments, the composition of the present invention is for use in treating inflammation.

In some embodiments, there is provided a composition for use in the treatment of inflammation in a subject in need thereof.

In one embodiment, the composition of the present invention can be provided to the individual per se. In one embodiment, the composition of the present invention can be provided to the individual as part of a pharmaceutical composition or a nutraceutical composition where it is mixed with a pharmaceutically or nutraceutically acceptable excipient.

In one embodiment, a “pharmaceutical composition” or a “nutraceutical composition” refers to a preparation of a composition as described herein with other chemical components such as physiologically suitable carriers and excipients.

In one embodiment, the phrases “physiologically acceptable carrier” and “pharmaceutically acceptable carrier” which can be interchangeably used refer to a carrier or a diluent that does not cause significant irritation to a mammal and does not abrogate the biological activity and properties of the administered composition. An adjuvant is included under these phrases.

In one embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington’s Pharmaceutical Sciences,” Mack Publishing Co., Easton, PA, latest edition, which is incorporated herein by reference in its entirety.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In one embodiment, the preparation is administered in a local rather than systemic manner, for example, via injection of the preparation directly into a specific region of a patient’s body. In one embodiment, the region of a patient’s body is characterized by inflammation or as comprising inflammatory mediators.

In one embodiment, the dosage of the composition of the present invention, is in the range of 0.5-2,000 mg/day. In another embodiment, the dosage is in the range of 5-500 mg/day. In another embodiment, the dosage is in the range of 500-2,000 mg/day. In another embodiment, the dosage is in the range of 0.1-10 mg/day. In another embodiment, the dosage is in the range of 50-500 mg/day. In another embodiment, the dosage is in the range of 5-4,000 mg/day. In another embodiment, the dosage is in the range of 0.5-50 mg/day. In another embodiment, the dosage is in the range of 5-80 mg/day. In another embodiment, the dosage is in the range of 100-1,000 mg/day. In another embodiment, the dosage is in the range of 1,000-2,000 mg/day. In another embodiment, the dosage is in the range of 200-600 mg/day. In another embodiment, the dosage is in the range of 400-1,500 mg/day. In another embodiment, the dosage is in a range of 800-1,500 mg/day. In another embodiment, the dosage is in the range of 500-2,500 mg/day. In another embodiment, the dosage is in a range of 600-1,200 mg/day. In another embodiment, the dosage is in the range of 1,200-2,400 mg/day. In another embodiment, the dosage is in the range of 40-60 mg/day. In another embodiment, the dosage is in a range of 2,400-4,000 mg/day. In another embodiment, the dosage is in a range of 450-1,500 mg/day. In another embodiment, the dosage is in the range of 1,500-2,500 mg/day. In another embodiment, the dosage is in the range of 5-10 mg/day. In another embodiment, the dosage is in the range of 550-1,500 mg/day.

In one embodiment, the dosage is at least 200 mg/day. In another embodiment, the dosage is at least 300 mg/day. In another embodiment, the dosage is at least 400 mg/day. In another embodiment, the dosage is at least 500 mg/day. In another embodiment, the dosage is at least 600 mg/day. In another embodiment, the dosage is at least 700 mg/day. In another embodiment, the dosage is at least 800 mg/day. In another embodiment, the dosage is at least 900 mg/day. In another embodiment, the dosage is at least 1,000 mg/day.

As used herein, the term “dosage” refers to the amount of an active ingredient or the combination of active ingredients of the invention. In another embodiment, “dosage” is not inclusive with respect to excipients. In some embodiments, an excipient is an aqueous solution, a buffer, a vehicle, or any other inert substance.

Oral administration, in one embodiment, comprises a unit dosage form comprising tablets, capsules, lozenges, chewable tablets, suspensions, drinks, syrups, nectars, beverages, emulsions and the like. Such unit dosage forms comprise a safe and effective amount of the composition. The pharmaceutically acceptable carriers suitable for the preparation of unit dosage forms for peroral administration are well-known in the art. In some embodiments, tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. In some embodiments, the selection of carrier components depends on secondary considerations like taste, cost, and shelf stability, which are not critical for the purposes of this invention, and can be readily made by a person skilled in the art.

In one embodiment, the oral dosage form comprises predefined release profile. In one embodiment, the oral dosage form of the present invention comprises an extended release tablets, capsules, lozenges or chewable tablets. In one embodiment, the oral dosage form of the present invention comprises a slow release tablets, capsules, lozenges or chewable tablets. In one embodiment, the oral dosage form of the present invention comprises an immediate release tablets, capsules, lozenges or chewable tablets. In one embodiment, the oral dosage form is formulated according to the desired release profile of the active ingredient as known to one skilled in the art. In another embodiment, the composition is a drink or a beverage comprising a dosage which consists a combination of the active ingredients in a ratio or in an amount as described herein.

Oral compositions, in some embodiments, comprise liquid solutions, emulsions, suspensions, and the like. In some embodiments, pharmaceutically acceptable carriers suitable for preparation of such compositions are well known in the art.

In one embodiment, a pharmaceutical composition of the present invention is manufactured by processes well known in the art, e.g., by means of conventional mixing, dissolving, granulating, dragee-making, levigating, emulsifying, encapsulating, entrapping or lyophilizing processes.

In one embodiment, a composition for use in accordance with the present invention is formulated in conventional manner using one or more physiologically acceptable carriers comprising excipients and auxiliaries, which facilitate processing of the active ingredients into preparations which, can be used pharmaceutically. In one embodiment, the formulation dependents upon the route of administration chosen.

In some embodiments, the composition further comprises a preservative selected from: benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases, to adjust the pH of the composition as needed.

In some embodiments, a composition suitable for use in context of the present invention comprises the active ingredient(s) in an amount effective to achieve the intended purpose, e.g., reduce inflammatory response. In some embodiments, a therapeutically effective amount means an amount of active ingredients effective to prevent, alleviate or ameliorate symptoms of disease or prolong the survival of the subject being treated.

In one embodiment, determination of a therapeutically effective amount is well within the capability of those skilled in the art.

In some embodiments, preparation of effective amount or dose can be estimated initially from in vitro assays. In one embodiment, a dose can be formulated in animal models and such information can be used to more accurately determine useful doses in humans.

Some examples of substances which can serve as nutraceutical or pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of Theobroma; polyols such as propylene glycol, glycerin, sorbitol, mannitol, and polyethylene glycol; alginic acid; emulsifiers, such as the Tween™ brand emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a nutraceutical or a pharmaceutically acceptable carrier to be used in conjunction with the compound is basically determined by the way the compound is to be administered. If the subject compound is to be injected, in one embodiment, the nutraceutical or pharmaceutically acceptable carrier is sterile, physiological saline, with a blood-compatible suspending agent, the pH of which has been adjusted to about 7.4.

In addition, the composition further comprises a binder (e.g., acacia, cornstarch, gelatin, carbomer, ethyl cellulose, guar gum, hydroxypropyl cellulose, hydroxypropyl methyl cellulose, povidone), a disintegrating agent (e.g., cornstarch, potato starch, alginic acid, silicon dioxide, croscarmellose sodium, crospovidone, guar gum, sodium starch glycolate), a buffer (e.g., Tris-HCl., acetate, phosphate) of various pH and ionic strength, an additive such as albumin or gelatin to prevent absorption to surfaces, detergents (e.g., Tween 20, Tween 80, Pluronic F68, bile acid salts), a protease inhibitor, a surfactant (e.g., sodium lauryl sulfate), a permeation enhancer, a solubilizing agent (e.g., glycerol, polyethylene glycerol), an anti-oxidant (e.g., ascorbic acid, sodium metabisulfite, butylated hydroxyanisole), a stabilizer (e.g., hydroxypropyl cellulose, hydroxypropylmethyl cellulose), a viscosity increasing agent (e.g., carbomer, colloidal silicon dioxide, ethyl cellulose, guar gum), a sweetener (e.g., aspartame, citric acid), a preservative (e.g., Thimerosal, benzyl alcohol, parabens), a lubricant (e.g., stearic acid, magnesium stearate, polyethylene glycol, sodium lauryl sulfate), a flow-aid (e.g., colloidal silicon dioxide), a plasticizer (e.g., diethyl phthalate, triethyl citrate), an emulsifier (e.g., carbomer, hydroxypropyl cellulose, sodium lauryl sulfate), a polymer coating (e.g., poloxamers or poloxamines), a coating and/or a film forming agent (e.g., ethyl cellulose, acrylates, polymethacrylates) and/or an adjuvant, or any combination thereof. Each possibility represents a separate embodiment of the invention.

Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g., AVICEL™, RC-591), tragacanth and sodium alginate; typical wetting agents include lecithin and polyethylene oxide sorbitan (e.g., polysorbate 80). Typical preservatives include methyl paraben and sodium benzoate. In another embodiment, peroral liquid compositions also contain one or more components such as sweeteners, flavoring agents and colorants disclosed above.

In one embodiment, toxicity and therapeutic efficacy of the composition described herein can be determined by standard nutraceutical or pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient’s condition. [See e.g., Fingl, et al., (1975) “The Pharmacological Basis of Therapeutics”, Ch. 1 p.1].

In one embodiment, depending on the severity and responsiveness of the condition to be treated, dosing can be of a single or a plurality of administrations, with course of treatment lasting from several days to several weeks or until cure is affected or diminution of the disease state is achieved.

In one embodiment, the amount of a composition to be administered depends on the subject being treated, the severity of the affliction, the manner of administration, the judgment of the prescribing physician, etc., or any combination thereof.

In one embodiment, a composition comprising the preparation of the present invention formulated in a compatible pharmaceutical or nutraceutical carrier may also be prepared, placed in an appropriate container, and labeled for treatment of an indicated condition.

In one embodiment, a composition of the present invention is presented in a pack or dispenser device, such as an FDA approved kit, which contain one or more unit dosage forms containing the composition. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of nutraceuticals or pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

According to some embodiments, the present invention provides a kit comprising: (a) a cannabinoid; and (b) lycopene.

According to some embodiments, the present invention provides a kit comprising: (a) a cannabinoid; (b) lycopene; and (c) mixing instructions for the cannabinoid and the lycopene.

In some embodiments, the cannabinoid comprises or is CBD or CBDA. In some embodiments, the cannabinoid comprises CBD and CBDA.

In some embodiments, the kit is for use in treatment of inflammation in a subject in need thereof.

In another embodiment, the kit further comprises: phytoene, phytofluene, beta carotene, tocopherol, a phytosterol, astaxanthin, lutein, a terpene, a polyphenol, or any combination thereof.

According to some embodiments, the kit is utilized by mixing a cannabinoid and lycopene and administering the composition formed by mixing the cannabinoid and lycopene, for example, by means of an oral route.

According to another embodiment, the kit is utilized by mixing: (a) a cannabinoid; (b) lycopene; and (c) a compound selected from: phytoene, phytofluene, beta carotene, tocopherol, a phytosterol, astaxanthin, lutein, a terpene, a polyphenol, or any combination thereof, and administering the composition formed, for example, by means of an oral route.

In some embodiments, the kit comprises instructions for mixing: (a) lycopene; and (b) a cannabinoid, in a m:m ratio ranging from 50:1 (m:m) to 1:10 (m:m), 50:1 (m:m) to 1:5 (m:m), 50:1 (m:m) to 1:2.5 (m:m), 50:1 (m:m) to 1:1 (m:m), 40:1 (m:m) to 1:5 (m:m), 30:1 (m:m) to 1:2 (m:m), 25:1 (m:m) to 1:2 (m:m), 20:1 (m:m) to 1:2.5 (m:m), 15:1 (m:m) to 1:1 (m:m), 10:1 (m:m) to 1:1.5 (m:m), 10:1 (m:m) to 1:3 (m:m), or 10:1 (m:m) to 1:1 (m:m). Each possibility represents a separate embodiment of the invention.

In some embodiments, the kit comprises instructions for mixing: (a) lycopene; and (b) CBD, in a m:m ratio ranging from 50:1 (m:m) to 5:1 (m:m), 40:1 (m:m) to 5:1 (m:m), 30:1 (m:m) to 5:1 (m:m), 20:1 (m:m) to 5:1 (m:m), 10:1 (m:m) to 5:1 (m:m), 7.5:1 (m:m) to 4:1 (m:m), 5:1 (m:m) to 1:1 (m:m), 25:1 (m:m) to 10:1 (m:m), or 20:1 (m:m) to 10:1 (m:m). Each possibility represents a separate embodiment of the invention.

In some embodiments, the kit comprises instructions for mixing: (a) lycopene; and (b) CBDA, in a m:m ratio ranging from 1:1 (m:m) to 1:50 (m:m), 1:1 (m:m) to 1:40 (m:m), 1:1 (m:m) to 1:30 (m:m), 1:1 (m:m) to 1:20 (m:m), 1:1 (m:m) to 1:15 (m:m), 1:1 (m:m) to 1:10 (m:m), 1:1 (m:m) to 1:5 (m:m), or 1:1 (m:m) to 1:4 (m:m), 1:1 (m:m) to 1:3 (m:m), 1:1 (m:m) to 1:2.5 (m:m), or 1:1 (m:m) to 1:2 (m:m). Each possibility represents a separate embodiment of the invention.

In some embodiments, the kit comprises instructions for mixing: (a) a cannabinoid; and (b) lycopene, in a weight per weight ratio ranging from 1:99 (w/w) to 99:1 (w/w), 1:80 (w/w) to 80:1 (w/w), 1:55 (w/w) to 55:1 (w/w), 1:30 (w/w) to 30:1 (w/w), or 1:10 (w/w) to 10:1 (w/w). Each possibility represents a separate embodiment of the invention.

In some embodiments of the subject kit, the cannabinoid and lycopene are packaged within a container. In some embodiments of the subject kit, the container further comprises a compound selected from: phytoene, phytofluene, beta carotene, tocopherol, a phytosterol, astaxanthin, lutein, a terpene, a polyphenol, or any combination thereof.

In some embodiments, the container is made of a material selected from: thinwalled film or plastic (transparent or opaque), paperboard-based, foil, rigid plastic, metal (e.g., aluminum), glass, etc.

In some embodiments, the content of the kit is packaged, as described below, to allow for storage of the components until they are needed.

In some embodiments, some or all components of the kit may be packaged in suitable packaging to maintain sterility.

In some embodiments of the subject kit, any one of: (a) a cannabinoid; (b) lycopene; and (c) a compound selected from: phytoene, phytofluene, beta carotene, tocopherol, a phytosterol, astaxanthin, lutein, a terpene, , a polyphenol, or any combination thereof, are stored in separate containers within the main kit containment element e.g., box or analogous structure, may or may not be an airtight container, e.g., to further preserve the sterility of some or all of the components of the kit.

In some embodiments, the dosage amount any one of: (a) a cannabinoid; (b) lycopene; and (c) a compound selected from: phytoene, phytofluene, beta carotene, tocopherol, a phytosterol, astaxanthin, lutein, a terpene, a polyphenol, or any combination thereof, provided in a kit may be sufficient for a single application or for multiple applications.

In some embodiments, the kit contains instructions for preparing the herein disclosed composition and for how to practice the method of the invention.

In some embodiments, the kit further comprises a measuring utensil such as syringe, measuring spoon, or a measuring cup.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Materials and Methods

### System Model - Lipopolysaccharide (LPS)-Challengedperitoneal Macrophages

Macrophage isolation and cell culture - Peritoneal macrophages were collected from the peritoneal cavity of 6-8-week-old male ICR mice (Harlan, Israel) given an intraperitoneal injection of 1.5 ml of thioglycollate broth (4%) 4 days before harvest. Peritoneal macrophages were washed three times with PBS and if needed a hypotonic lysis of erythrocytes was performed, yielding 90-95% purity. The macrophages were identified by fluorescence-activated cell sorting (FACS) analysis using FITC-conjugated rat antimouse F4/80 (MCA497F; Serotec, Oxford, England) by flow microfluorimetry on FACS (Becton Dickinson, Mountain View, CA). For each sample, 10,000 light scatter-gated viable cells were analyzed. Peritoneal macrophages and murine macrophage cell line RAW264.7 were cultured RPMI 1640 medium containing 10% FCS, 2 mM L-glutamine; 100 U/ml penicillin; 100 µg/ml streptomycin (Beit-Haemek, Israel) in 96-well plates (1 × 10⁶ cells/well) at 37° C. in 5% CO₂ atmosphere. Cells were stimulated with LPS (1 µg/ml) in the presence or absence of the food lycopene, or CBD and their combination.

The ingredients were dissolved in DMSO (in a final concentration of 5 mM). The mixture is vortexed ruggedly and shacked at 37° C. for 10 min and sonicated in a sonicator bath for 15 sec × 3 times. From this stock solution the desired concentrations are obtained by addition of appropriate volumes of warm culture medium.

The concentration in solution was calculated to 1 ml of the highest final concentration 0.5 ml isopropanol + 1.5 hexane/dichloromethane (1:5 v/v) containing 0.025% BHT. The solution is vortexed, and the phases were separated by centrifugation at 3,000 rpm for 10 min.

A spectral analysis is performed to detect the level of lycopene with the characteristic peak at 471 nm.

To the control groups, appropriate volumes of DMSO (0.1-0.2%) were added. The percent inhibition of each experimental group was calculated comparatively to the relevant and corresponding control.

NO production assay —NO levels in supernatants of cell cultures were determined by assaying nitrite levels using Griess reagent and sodium nitrite as a standard.

Statistical significance for comparisons between groups was determined using Student’s paired two-tailed t test. Data are presented as the mean ± standard error of the mean (SEM).

Peritoneal macrophages were incubated with the compounds 1 h before addition of LPS and cultured overnight. NO production was determined in the supernatant of the cultures and the % of inhibition from untreated macrophages was determined.

Series of experiments included the following: dose dependent of NO production inhibition by lycopene (in a molar concentration ranging from 0.1-5 µM); dose dependent of NO production inhibition by CBD (in a molar concentration ranging from 1-10 µM); and the effect of combinations of lycopene and CBD (Lycopene + CBD) on NO production inhibition.

### System Model - LPS-Challenged Juman Monocytes Isolated from Blood

Bone marrow derived macrophages were treated with an inflammatory stimulus in the presence of various concentration of lycopene, cannabidiolic acid (CBDA), tomato extract and hemp extract in vitro. As an inflammatory outcome, nitric oxide (NO) was monitored.

Prescreen of test material (Safety/Viability testing) - Cells were cultured with various concentrations of the test material, solvent, and benchmarks to determine a safe range of concentrations for use in the subsequent study. Serial dilutions of lycopene and Hemp extracts (5 concentrations in triplicates) were tested. The cells were incubated with the materials for 24 h or overnight and at the end of the treatment period changes in cell viability were determined using an XTT/MTT/LDH assay.

Full Study with test material and benchmarks - Cells were treated with tomato and hemp extract for 24 hours to determine viability. Cells were then treated as follows; (1) medium alone (unstimulated control), (2) LPS stimulation- 10 µg/mL, (3) each crude extracts (Tomato/Hemp) at concentration of IC50, (4) LPS + crude extracts, (5) control inhibitor, and (6) LPS + control inhibitor. The tomato and hemp extract and benchmarks (lycopene, CBDA) were tested in 5 different rations; 1:10, 1:5, 1:1, 5:1, 10:1.

Nitric oxide production was determined for each treatment.

### Example 1

### Anti-Inflammatory Synergistic Effect of Lycopene and a Cannabinoid

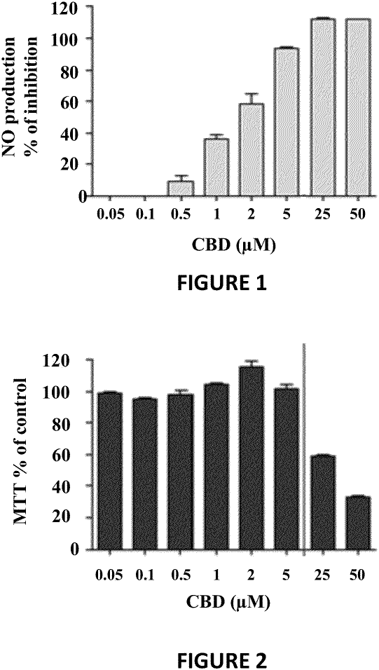

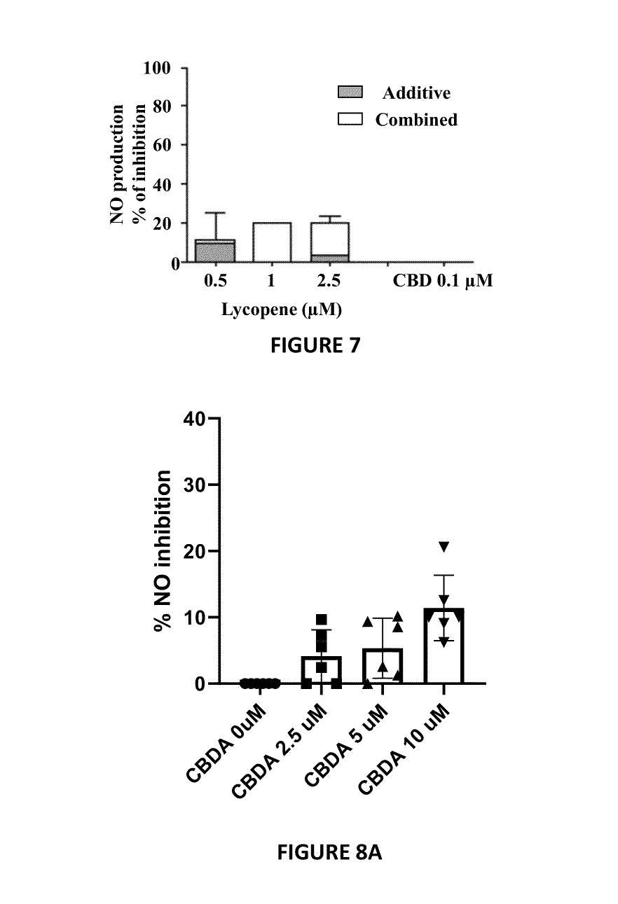

The inventors initially examined the sole effect of cannabidiol (CBD) on nitric oxide (NO) production. Macrophages were stimulated, and NO production was examined in the presence of increasing CBD concentrations, ranging from 0.05 µM to 50 µM. The inventors found that the % of NO production inhibition increased in a CBD dose-dependent manner (FIG. 1). Nonetheless, CBD at a concentration of 25 µM or more, was found to be highly toxic to the cells (FIG. 2).

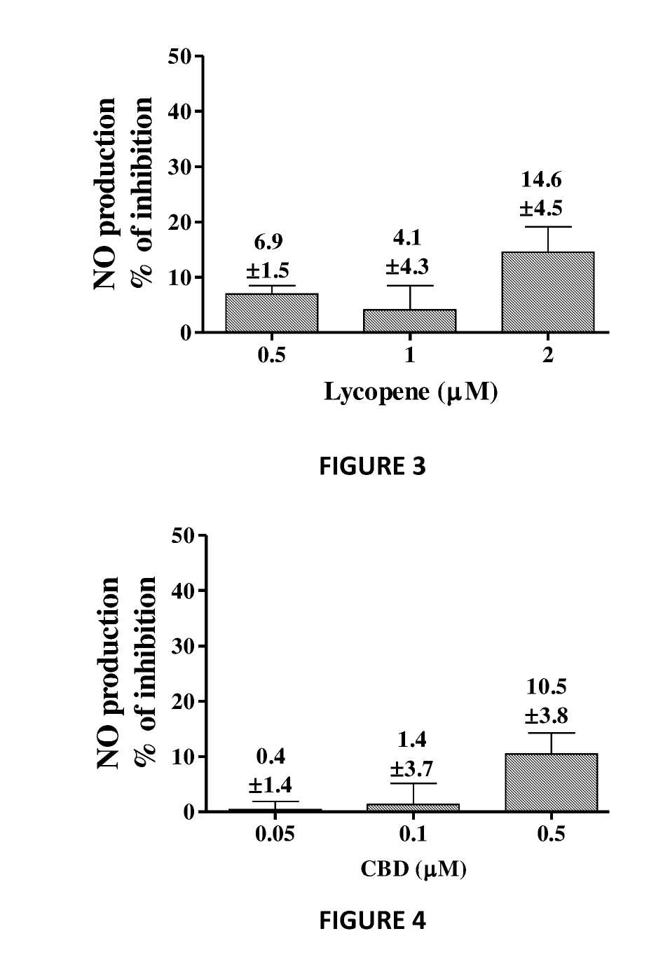

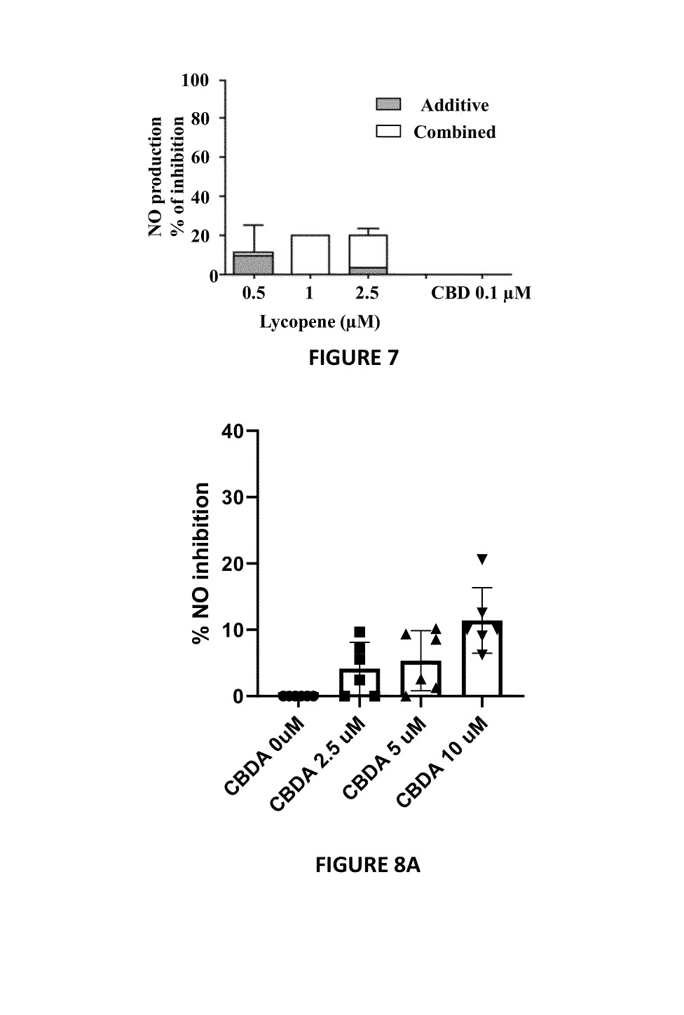

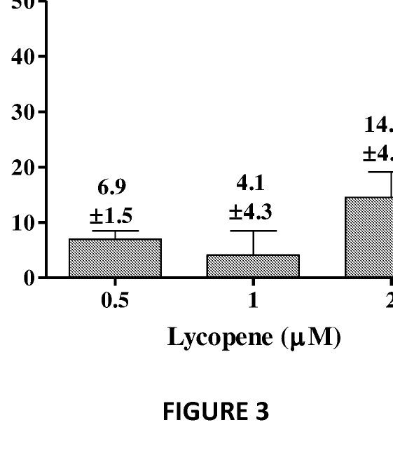

Accordingly, for examining whether a synergistic anti-inflammatory effect exists for lycopene and CBD, the inventors treated stimulated macrophages with CBD at a concentration of 0.05 µM to 0.5 µM (the sole effect is depicted in FIG. 4) and lycopene at a concentration ranging from 0.5 µM to 2 µM (the sole effect is depicted in FIG. 3).

The inventors showed that the addition of 0.05 µM CBD to lycopene at a concentration ranging from 0.5 µM to 1 µM, increased the inhibitory effect on NO production by approximately 10-15% (FIG. 5). Further, inventors showed that the addition of 0.1 µM CBD to lycopene at a concentration ranging from 1 µM to 2 µM, increased the inhibitory effect on NO production by approximately 25% (FIG. 6).

Further, the inventors examined whether a combination of lycopene and CBDA also harbors an anti-inflammatory effect. The inventors showed that CBDA inhibits NO production in a dose dependent manner (FIG. 8A). Further, the inventors showed that the addition of 2.5 µM CBDA to lycopene at a concentration of 1 µM, increased the inhibitory effect on NO production by a 6-fold (FIG. 8B).

### Example 2

CBD is mixed with Lycomato, astaxanthin, or a combination thereof. Lycomato is tomato oleoresin comprising: lycopene 6% or 7% (w/w), phytoene and phytofluene 1.5% (w/w), tocopherols 1.5% (w/w), phytosterols 2% (w/w) and lipids from tomato. The composition is tested as follows: (a) in vitro - TNFα assay on LPS induced-THP monocyte cells; (b) in vivo - acute dextran sulfate sodium (DSS) colitis mice model; and Chronic IL-10 knockout mode; and (c) IBD/IBS clinical study. This combination is directed to the promotion of health and function of the digestive system.

### Example 3

Lycomato 6% or 7% (as described above) or Cardiomato that is tomato oleoresin comprising lycopene to phytosterols ratio of 1:1 (5.8% (w/w): 5.8% (w/w)) are mixed with CBD. The composition is tested as follows: (a) in vitro - TNFα assay on LPS induced-THP monocyte cells; (b) in vivo - ApoE knockout mode; and (c) A clinical study with an endpoint of endothelial function. This combination is directed to the promotion of health and wellbeing of the arteries and cardiovascular system.

### Example 4

CBD is mixed with Lycomato 6% or 7% (as described above), lutein, astaxanthin, or a combination thereof. The composition is tested as follows: (a) in vitro - secretion of pro-inflammatory mediators from microglia brain cells; (b) in vivo - mice inoculated with human Aβ42 peptide; mice intraventricularly injected with fibrillar Aβ; and APP × PS1 transgenic mice; and (c) A clinical study with an endpoint of symptomatic relief in anxiety, spatial disorientation, and memory function. This combination is directed to the memory boost, reduction of anxiety, and improved spatial coordination for elderly (e.g., third age seniors).

### Example 5

CBD is mixed with Lycomato DAL or Lycomato CTC. Lycomato DAL is tomato oleoresin with less than 1% (w/w) of lycopene and other phytonutrients as in Lycomato 6%. CTC is 12 times concentrated tomato serum (water soluble compounds). CTC contains ketosamines. The composition is tested as follows: (a) in vitro - modulation of inflammatory processes in a macrophage immune cell model; (b) A clinical study (e.g., open label study) with an endpoint of symptomatic relief in joint pain and mobility. This combination is directed to the promotion of flexibility and health of joints and cartilage tissue.

### Example 6

CBD is mixed with Lycomato (as described above), Lycomato DAL (as described above) or golden tomato. Golden tomato comprises phytoene and phytofluene 6-8% (w/w); zeta-carotene 1.5-2% (w/w); lycopene 0.1% (w/w), tocopherols 2.5% (w/w), phytosterols ~1% (w/w) and lipids from the tomato. The composition is tested as follows: (a) in vitro -hormonal balance and antioxidant response in breast, uterine, or bone cells activated by estrogens; (b) A clinical study (e.g., open label study) with an endpoint of symptomatic relief in menstrual discomfort and hormonal balance. This combination is directed to the promotion of reduce mensuration pain, discomfort and weakness, and relieve post menstrual syndrome (PMS) symptoms.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A composition comprising a cannabinoid and lycopene.

2. The composition of claim 1, wherein the molar ratio (m:m) of said lycopene to said cannabinoid is from 50:1 (m:m) to 1:10 (m:m).

3. The composition of claim 1, wherein said cannabinoid is selected from the group consisting of: tetrahydrocannabinol (THC), iso-tetrahydrocannabinol- type (iso-THC), tetrahydrocannabinolic acid (THCA), cannabidiol (CBD), cannabidiolic acid (CBDA), cannabinol (CBN), cannabinolic acid (CBNA), cannabinol methyl ether (CBNM), cannabinol-C4 (CBN—C4), cannabinol-CZ (CBN—C2), cannabiorcol (CBN—C1), cannabinodiol (CBND), cannabigerol (CBG), cannabigerolic acid (CBGA), cannabigerolic acid monomethyl ether (CBGAM), cannabigerol monomethyl ether (CBGM), cannabigerovarinic acid (CBGVA), cannabichromene (CBC), cannabichromanon (CBCN), cannabichromenic acid (CBCA), cannabichromevarin (CBCV), cannabichromevarinic acid (CBCVA), tetrahydrocannabivarin (THCV), cannabidivarin (CBDV), cannabielsoin (CBE), cannabielsoic acid A (CBEA—A), cannabielsoic acid B (CBEA—B), cannabigerovarin (CBGV), cannabidiolic acid (CBDA), cannabidiol monomethyl ether (CBDM), cannabidiol- C4 (CBD—C4), cannabidivarinic acid (CBDVA), and cannabidiorcol (CBD—C1), cannabicyclol (CBL), cannabicyclolic acid (CBLA), cannabicyclovarin (CBLV), cannabitriol, cannabitriolvarin (CBTV), ethoxy-cannabitiolvarin (CBTVE), cannabivarin (CBV), cannabidivarin (CBVD), cannabitriolvarin (CBTV), ethoxy-cannabitiolvarin (CBTVE), cannabifuran (CBF), dehydrocannabifuran (DCBF), and cannabiripsol (CBR), or any combination thereof.

4. The composition of claim 1, wherein said cannabinoid is selected from the group consisting of: CBD, (-)-7-hydroxy-CBD, (-)-CBD-7-oic acid, dimethylheptyl homologue of (-)-7-hydroxy-CBD, dimethylheptyl homologue of (-)-CBD-7-oic acid, or any combination thereof.

5. The composition of claim 1, wherein said cannabinoid is CBD or CBDA.

6. The composition of claim 5, comprising said lycopene and said CBD in a m:m ratio ranging from 30:1 (m:m) to 10:1 (m:m).

7. The composition of claim 5, comprising said lycopene and said CBDA in a m:m ratio ranging from 1:5 (m:m) to 1:1 (m:m).

8. The composition of claim 1, further comprising: phytoene, phytofluene, beta carotene, tocopherol, a phytosterol, astaxanthin, lutein, a terpene, a polyphenol, or any combination thereof.

9. The composition of claim 1, further comprising: phytoene, phytofluene, or any combination thereof.

10. The composition of claim 1, wherein said composition is an oral composition.

11. The composition of claim 1, wherein said cannabinoid is present as a highly purified extract of Cannabis.

12. The composition of claim 1, wherein said cannabinoid is a synthetically produced cannabinoid.

13. A pharmaceutical composition, comprising the composition of claim 1 and a pharmaceutically acceptable carrier.

14. (canceled)

15. A method for treating a subject afflicted with inflammation, comprising administering to said subject a therapeutically effective amount of the composition of claim 1.

16. The method of claim 15, wherein said treating comprises reducing or inhibiting production of nitric oxide in said subject.

17. A kit comprising: (a) lycopene; (b) a cannabinoid; and (c) mixing instructions for said lycopene and said cannabinoid.

18. The kit of claim 17, comprising instructions for administering said lycopene and said cannabinoid in a m:m ratio ranging from 50:1 (m:m) to 1:10 (m:m).

19. The kit of claim 17, wherein said cannabinoid is present as a highly purified extract of Cannabis.

20. The kit of claim 17 , wherein said cannabinoid is a synthetically produced cannabinoid.

21. The kit of claim 17, wherein said cannabinoid comprises CBD or CBDA.

22. (canceled)

23. (canceled)
